(19) United States
(12) Patent Application Publication
(10) Pub. No.: US 2006/0237007 A1
(43) Pub. Date: Oct. 26, 2006

(54) Pack for holding resuscitation facemask

(75) Inventor: Johnny R. E. Quest, Ocoee, FL (US)

Correspondence Address: WONG, CABELLO, LUTSCH, RUTHERFORD & BRUCCULERI,;L.L.P., 20333 SH 249, SUITE 600, HOUSTON, TX, 77070, US

(73) Assignee: Jeff Ellis & Associates, Inc., Kingwood, TX (US)

(21) Appl. No.: 11/112,052

(22) Filed: Apr. 22, 2005

(51) Int. Cl. A61M 16/00

(52) U.S. Cl. 128/203.11

(57) ABSTRACT

A pack worn by a rescuer holds an assembled resuscitation facemask. The pack has a pouch and has a flap and belt attached to the pouch. A face portion of the mask fits into an opening in the pouch. Preferably, a cutaway along the edge of the opening accommodates the projecting mouthpiece of the facemask. The flap closes over the mask in the opening, and an edge of the flap fastens to the pouch. The flap defines a cutout for the mouthpiece to project from the flap. Preferably, the cutout in the flap connects with a split defined in an edge of the flap. The split is smaller than the cutout such that first and second portions of the flap fit around the projecting mouthpiece when the flap is closed and fastened to the pouch.

### FIELD OF THE INVENTION

The subject matter of the present disclosure relates to a pack for holding a resuscitation facemask.

### BACKGROUND OF THE INVENTION

Resuscitation facemasks are used by rescuers, such as lifeguards, to resuscitate patients. Referring to FIG. 1, a prior art resuscitation facemask 10 is illustrated. The facemask 10 is similar to the commercially available Seal Easy™ by Respironics, Inc. The facemask 10 has a face portion 12, which is an inflated cushion made of an elastomeric material. The face portion 12 has an opening 13 through it. A plastic tube 16 projects from the face portion 12 and communicates with the opening 13. A one-way valve 18, which is removable, fits into the end of the projecting tube 16. The projecting tube 16 and valve 18 form a mouthpiece 14 for the rescuer to use when resuscitating a patient. Prior to use, the one-way valve 18 is pushed into the end of the projecting tube 16 to create the assembled facemask. Once assembled, the inflated cushion of the face portion 12 is positioned over a patient's face with the opening 13 in the cushion positioned at the patient's mouth. The cushion of the face portion 12 forms a secure seal on the face of the patient, and the rescuer uses the mouthpiece 14 to direct air into the patient's lungs. The one-way valve 18 directs the patients exhaled gases away from the rescuer and reduces the chances of cross-contamination between rescuer and patient.

A rescuer, such as a lifeguard, typically carries the resuscitation facemask 10 in a conventional hip pack 20 of the prior art, which is also shown in FIG. 1. The prior art hip pack 20 has a pouch 22, a cover 24, and a belt 26. One way of holding the facemask 10 in the pack 20 involves separately placing the disassembled components of the facemask 10 into the pouch 22. This has the disadvantage of requiring a rescuer to assemble the facemask 10 during a rescue and creates the risk of losing one of the components parts.

Another way of holding the facemask 10 in the pack 20 involves pre-assembling the valve 18 on the projecting tube 16 and placing the assembled facemask 10 in the pouch 22 with the mouthpiece 14 projecting out of the zippered opening. Then, the two zippers 25 for the cover 24 can be closed to meet on either side of the projecting mouthpiece 14. Unfortunately, the zippers 25 cannot properly hold the facemask 10 in the pouch 22 because movement of the mouthpiece 14 can cause the zippers 25 to open. For example, the facemask 10 has the potential of becoming unsecured from the pouch 22 when a lifeguard wearing the hip pack 20 enters the water with the mouthpiece 14 held by the zippers 25.

Yet another way of holding the facemask 10 in the pack 20 involves fitting the assembled facemask 10 within the pouch without the mouthpiece 14 projecting out. The zippers 25 for the cover 24 can then be fully closed. Unfortunately, the facemask 10 must be stored sideways in the pouch 22, making the pouch 22 bulky. Furthermore, there is greater potential of breaking the facemask 10 or catching the elastomeric material of the face portion 12 in the zippers 25 causing it to rupture.

Consequently, a need exists for a device to hold a resuscitation facemask securely while the facemask remains assembled and the device is worn by a rescuer. The subject matter of the present disclosure is directed to overcoming, or at least reducing the effects of, one or more of the problems set forth above.

### SUMMARY OF THE DISCLOSURE

A pack worn by a rescuer holds an assembled resuscitation facemask. The pack has a pouch and has a flap and belt attached to the pouch. A face portion of the mask fits into an opening in the pouch. Preferably, a cutaway along the edge of the opening accommodates the projecting mouthpiece of the facemask. The flap closes over the mask in the opening, and an edge of the flap fastens to the pouch. The flap defines a cutout for the mouthpiece to project from the flap. Preferably, the cutout in the flap connects with a split defined in an edge of the flap. The split is smaller than the cutout such that first and second portions of the flap fit around the projecting mouthpiece when the flap is closed and fastened to the pouch.

The foregoing summary is not intended to summarize each potential embodiment or every aspect of the present disclosure.

### BRIEF DESCRIPTION OF THE DRAWINGS

The foregoing summary, preferred embodiments, and other aspects of subject matter of the present disclosure will be best understood with reference to a detailed description of specific embodiments, which follows, when read in conjunction with the accompanying drawings, in which:

FIG. 1 illustrates a resuscitation facemask and a hip pack according to the prior art.

FIG. 2 illustrates an embodiment of a pack for holding a resuscitation facemask according to certain teachings of the present disclosure.

FIG. 3 illustrates the disclosed pack holding the facemask.

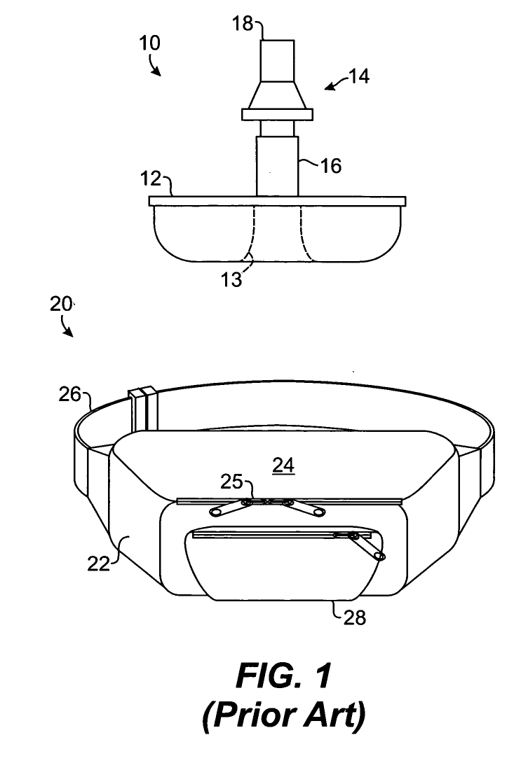

While the disclosed pack for holding a resuscitation facemask is susceptible to various modifications and alternative forms, specific embodiments thereof have been shown by way of example in the drawings and are herein described in detail. The figures and written description are not intended to limit the scope of the inventive concepts in any manner. Rather, the figures and written description are provided to illustrate the inventive concepts to a person skilled in the art by reference to particular embodiments, as required by 35 U.S.C. § 112.

### DETAILED DESCRIPTION

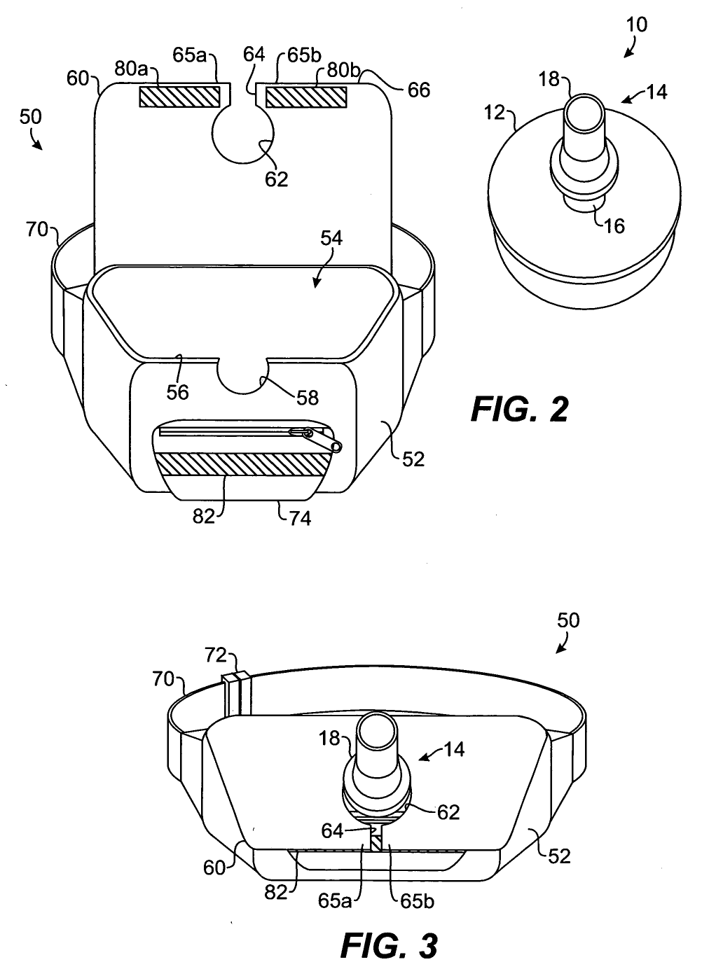

Referring to FIGS. 2 and 3, an embodiment of a pack 50 for holding a resuscitation facemask 10 is illustrated according to certain teachings of the present disclosure. The pack 50 includes a pouch 52 and a flap 60. The pack 50 can be made of any suitable material. Preferably, the pouch 52 has a strap or belt 70 attached for fitting the pouch 52 on a rescuer, and the belt 70 also preferably has a buckle 72. Alternatively, the pouch 52 has loops or slits (not shown) through which a belt worn by a rescuer can be threaded. The pouch 52 can also have a second compartment 74 for holding various

In FIG. 2, the flap 60 is shown open, and the facemask 10 is shown removed form the pouch 52. In FIG. 3, the facemask 10 is shown positioned in the pouch 52, and the flap 60 is shown closed. The facemask 10 is similar to the commercially available Seal Easy™ by Respironics, Inc. The facemask 10 has a face portion 12, a plastic tube 16 projects from the face portion 12, and a removable one-way valve 18 fits into the end of the projecting tube 16. The projecting tube 16 and valve 18 form a mouthpiece 14 for the rescuer to use when resuscitating a patient.

As best shown in FIG. 2, the pouch 52 defines an opening 54 for fitting the face portion 12 of the facemask 10 into the pouch 52. When the face portion 12 is positioned in the pouch 52, the mouthpiece 14 of the facemask 10 is allowed to project from the opening 54. Preferably, an edge 56 of the opening 54 defines a cutaway 58 to accommodate the projecting mouthpiece 14.

The flap 60 is attached to the pouch 52 for covering the opening 54 and keeping the facemask 10 in the pouch 52. The flap 60 defines a cutout 62 to allow the mouthpiece 14 to project from the flap 60 when the flap 60 is closed over the opening 54. The cutout 62 defined in the flap 60 preferably connects with a split 64 defined in an edge 66 of the flap 60. As best shown in FIG. 3, the split 64 defined in the edge 66 is preferably smaller than the cutout 62 such that first and second portions 65a-b of the flap 60 fit around the projecting mouthpiece 14 when the flap 60 is closed.

Fasteners 80 and 82 are respectively attached to the pouch 52 and the flap 60 to keep the flap 60 closed against the pouch 52. Any suitable fasteners can be used. For example, snaps, buttons, buckles, or the like can be used. Preferably, the fasteners 80 and 82 are corresponding portions of VELCRO because VELCRO offers secure fastening, can be readily undone by a rescuer, and can be easily resealed. First VELCO portions 80a-b are attached to the first and second flap portions 65a-b near the edge 66 of the flap. The VELCRO portions 80a-b are preferably sewn to the material of the flap 60. A second VELCRO portion 82 is attached to the pouch 52. Preferably, the VELCRO portion 82 is sewn to the material of the pack 20. In the present embodiment, the VELCRO portion 82 is a long piece of VELCO sewn to the second compartment 74 provided on the pouch 52. Ends of the VELCRO portion 82 are preferably sewn in to the edges of material used to form the second compartment 74 to prevent loosening of the VELCRO portion 82. When the flap 60 is closed, the VELCRO portions 80a-b and 82 are capable of fastening together to keep the flap 60 closed and the facemask 10 in the pack 50.

Using the belt 70, the pack 50 can be worn on the waist of a rescuer, such as a lifeguard. The pack 50 secures the facemask 10 to the rescuer while allowing the mouthpiece 14 to remain properly assembled for use. A tug on the flap 60 releases the VELCRO fasteners 80a-b and 82 to allow the rescuer to open the flap 60 and remove the assembled facemask 10 from the pouch 52.

The foregoing description of preferred and other embodiments is not intended to limit or restrict the scope or applicability of the inventive concepts conceived of by the Applicants. In exchange for disclosing the inventive concepts contained herein, the Applicants desire all patent rights afforded by the appended claims. Therefore, it is intended that the appended claims include all modifications and alterations to the full extent that they come within the scope of the following claims or the equivalents thereof.

What is claimed is:

1. A pack for holding a resuscitation facemask for a rescuer, the facemask having a projecting mouthpiece, the pack comprising:
a pouch defining an opening, the pouch holding the facemask and allowing the mouthpiece to project from the opening;
a first fastener attached to the pouch;
a flap attached to the pouch for covering the opening, the flap defining a cutout for the mouthpiece to project from the flap; and
a second fastener attached to the flap and attaching to the first fastener on the pouch to substantially hold the flap closed.

2. The pack of claim 1, further comprising a strap attached to the pouch for holding the pouch on the rescuer.

3. The pack of claim 1, wherein the first and second fasteners comprise portions of VELCRO.

4. The pack of claim 1, wherein the cutout defined in the flap connects with a split defined in an edge of the flap.

5. The pack of claim 4, wherein the split defined in the edge is smaller than the cutout such that first and second portions of the flap are capable of fitting around the projecting mouthpiece when the flap is closed.

6. The pack of claim 1, wherein an edge of the opening of the pouch defines a cutaway for the projecting mouthpiece.

7. A system worn by a rescuer for resuscitating a patient, the system comprising:
a facemask having a projecting mouthpiece;
a pouch defining an opening, the pouch holding the facemask and allowing the mouthpiece to project from the opening;
a first fastener attached to the pouch;
a flap attached to the pouch for covering the opening, the flap defining a cutout for the mouthpiece to project from the flap; and
a second fastener attached to the flap and attaching to the first fastener on the pouch to substantially hold the flap closed.

8. The system of claim 7, further comprising a strap attached to the pouch for holding the pouch on the rescuer.

9. The system of claim 7, wherein the first and second fasteners comprise portions of VELCRO.

10. The system of claim 7, wherein the cutout defined in the flap connects with a split defined in an edge of the flap.

11. The system of claim 10, wherein the split defined in the edge is smaller than the cutout such that first and second portions of the flap are capable of fitting around the projecting mouthpiece when the flap is closed.

12. The system of claim 7, wherein an edge of the opening of the pouch defines a cutaway for the projecting mouthpiece.

13. A system worn by a rescuer for resuscitating a patient, the system comprising:
a facemask having a projecting mouthpiece;
a pouch defining an opening, the pouch holding the facemask and allowing the mouthpiece to project from the opening;
a first fastener attached to the pouch;
a flap attached to the pouch for covering the opening, the flap defining a cutout and a split, the cutout allowing the mouthpiece to project from the flap, the split defined in an edge of the flap and connecting with the cutout such that first and second portions of the flap are capable of fitting around the projecting mouthpiece when the flap is closed; and
a second fastener attached to the flap and attaching to the first fastener on the pouch to substantially hold the flap closed.

14. The pack of claim 13, further comprising a strap attached to the pouch for holding the pouch on the rescuer.

15. The pack of claim 13, wherein the first and second fasteners comprise portions of VELCRO.

16. The pack of claim 13, wherein an edge of the opening of the pouch defines a cutaway for the projecting mouthpiece.
